(19) United States
(12) Patent Application Publication
(10) Pub. No.: US 2006/0040314 A1
(43) Pub. Date: Feb. 23, 2006

(54) Methods for screening polypeptides

(75) Inventors: Fred Christians, Los Altos Hills, CA (US); Kyle B. Cole, Stanford, CA (US)

Correspondence Address: AFFYMETRIX, INC;ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA (US)

(21) Appl. No.: 11/260,989

(22) Filed: Oct. 28, 2005

Related U.S. Application Data
(63) Continuation of application No. 09/683,613, filed on Jan. 24, 2002, now abandoned.
(60) Provisional application No. 60/264,635, filed on Jan. 25, 2001.

(51) Int. Cl. C12Q 1/68; C40B 40/08

(52) U.S. Cl. 435/6

(57) ABSTRACT

In one aspect of the invention, methods are provided for the creation and screening of polypeptides that eliminates bacterial cloning and individual screening In preferred embodiments, the method involves partnering each protein with a unique DNA oligonucleotide tag that directs the protein to a unique site on the microarray due to specific hybridization with a complementary tag-probe on the array

### CROSS REFERENCE TO RELATED APPLICATIONS

This application claims the priority of U.S. Provisional Application Ser. No. 60/264,635, titled “High Density GeneChip® Oligonucleotide Probe Array,” filed on Jan. 25, 2001, attorney docket number 3386 The '635 application is incorporated herein by reference in its entirety for all purposes

### BACKGROUND OF INVENTION

This invention relates to polypeptide screening using microarrays

High-density DNA probe arrays provide a highly parallel approach to nucleic acid sequence analysis that is transforming gene-based biomedical research Photolithographic DNA synthesis has enabled the large-scale production of GeneChip® probe arrays containing hundreds of thousands of oligonucleotide sequences on a glass chip typically about 15 cm² in size The manufacturing process integrates solid-phase photochemical oligonucleotide synthesis with lithographic techniques similar to those used in the microelectronics industry Due to their very high information content, GeneChip probe arrays are finding widespread use in the hybridization-based detection and analysis of mutations and polymorphisms (genotyping), and in a wide range of gene expression studies

### SUMMARY OF INVENTION

In one aspect of the invention, methods are provided for the creation and screening of polypeptides that eliminates bacterial cloning and individual screening In preferred embodiments, the method involves partnering each protein with a unique DNA oligonucleotide tag that directs the protein to a unique site on the microarray due to specific hybridization with a complementary tag-probe on the array Oligonucleotide tag arrays are also disclosed in, for example, U.S. patent application Ser. No. 09/746,036, Attorney Docket Number 3366 1, filed on Dec. 21, 2001

A mixture of thousands of different tag-protein pairs can then be screened for activity simultaneously, and proteins with desired activities can be identified by their position on the microarray

FIG. 14 illustrates one way in which a microarray with tag-probes could be used to screen a protein library, with no cloning needed To a protein-encoding mRNA a 5″ tag sequence and a 3″ ribosome-blocking sequence are attached (A) In a pool of such molecules, such as a randomly mutated gene library, each mRNA is paired with a unique tag and all have the same 3″ sequence Following in-vitro translation either on a microarray or in a test tube, the nascent protein remains attached to the mRNA (B), as in the technique of ribosome display (see, e g, Hanes, et al (2000) Methods Enzymol 328 404) During hybridization the tag directs each mRNA or mRNA-protein complex to a particular address on the Tag probe array (C), where all the proteins are screened simultaneously for activity (D) Appropriate detection methods identify proteins of interest (E), and the corresponding tag is known by the address on the array Finally, the corresponding genes can be captured by RT-PCR of the mRNA pool, either from the mRNA on the array or from another aliquot, using a universal reverse primer and each identified Tag sequence as a forward primer The genes can then be subjected to further screening or another round of mutagenesis

In another aspect of the invention, the tag system is used to screen (poly) peptides made from existing mRNA molecules for properties such as drug binding For example, all the mRNAs from a pathogenic bacterial strain could be made into tagged proteins which would be screened for the ability to bind antibiotic candidates The RNA molecules themselves could also be screened, as some drugs act directly on RNA The oligonucleotide tag could also be added directly to proteins, a method that is useful in cases in which clones are already separated and one wishes to use the tag probe array only for parallel screening

### BRIEF DESCRIPTION OF DRAWINGS

The accompanying drawings, which are incorporated in and form a part of this specification, illustrate embodiments of the invention and, together with the description, serve to explain the principles of the invention

FIG. 1 GeneChip® System Overview

FIG. 2 Wafer-scale GeneChip production specifications

FIG. 3 Photolithographic synthesis of oligonucleotide arrays

FIG. 4 Chemical preparation of glass substrates for light-directed synthesis of oligonucleotide arrays

FIG. 5 Automated array manufacturing

FIG. 6 Light-directed oligonucleotide synthesis cycle using MeNPOC photolabile phosphoramidite building blocks

FIG. 7 Method for fluorescent labeling and cleavage of photolithographically synthesized oligonucleotides allows quantitative analysis by HPLC

FIG. 8 Alternate photoremovable protecting groups for photolithographic oligonucleotide synthesis

FIG. 9 DNA probe array synthesis using photoacid generation in a polymer film to remove acid-labile DMT protecting groups

FIG. 10 Gene expression monitoring with oligonucleotide arrays A An image of a hybridized 1 28×1 28 cm HuGeneFL array, with 20 probe pairs for each of approximately 5000 full-length human genes B Probe design To control for background and cross-hybridization, each perfect match probe is partnered with a probe of the same sequence except containing a central mismatch Probes are usually 25mers, and are generally chosen to interrogate the 3″ regions of eukaryotic transcripts to mitigate the consequences of partially degraded mRNA

FIG. 11 Resequencing array for sequence variation detection A Each base of a given reference sequence is represented by four probes, usually 20mers, that are identical to each other with the exception of a single centrally located substitution (bold) Shown are probe sets targeted to two adjacent positions of the reference sequence B The target sequence is determined by hybridization intensities, with the probe complementary to the target providing the strongest signal

FIG. 12 HuSNP array design A A known biallelic polymorphism at position 0 is interrogated by a block of four or five probe sets (five in this example) Each probe set consists of four probes, a perfect match and a mismatch to allele A, and a perfect match and a mismatch to allele B One probe set in a block is centered directly over the polymorphism (0), and others are centered upstream (−4, −1) and downstream (+1, +4) B The sequences of the probe set centered over the polymorphism is shown C Sample images of blocks showing homozygous A, heterozygous A/B, or homozygous B at the same SNP site

FIG. 13 Schematic of the single-base extension assay applied to Tag probe arrays Regions containing known SNP sites (A or G in this example) are first amplified by PCR The PCR product serves as the template for an extension reaction from a chimeric primer consisting of a 5″ tag sequence and a 3″ sequence that abuts the polymorphic site The two dideoxy-NTPs that could be incorporated are labeled with different flurophors, in this example ddUTP is incorporated in the case of the A allele, and ddCTP for the G allele Multiple SBE reactions can be done in a single tube The tag sequence, unique for each SNP, directs the extension products to a particular address on the Tag probe array The proportion of a fluorophor at an address reflects the abundance of the corresponding allele in the original DNA

FIG. 14 Using Tag probe arrays to screen protein activity To a protein-encoding mRNA a 5″ tag sequence and a 3″ ribosome-blocking sequence are attached (A) In a pool of such molecules, such as a randomly mutated gene library, each mRNA is paired with a unique tag and all have the same 3″ sequence Following in-vitro translation either on a microarray or in a test tube, the nascent protein remains attached to the mRNA (B) During hybridization the tag directs each mRNA-protein to a particular address on the Tag probe array (C), where all the proteins are screened simultaneously for activity (D) Appropriate detection methods identify proteins of interest (E, black and/or shaded blocks) Finally, the corresponding genes can be captured by PCR of the mRNA pool using a universal reverse primer and each identified Tag sequence as a forward primer

FIG. 15 PCR based method for attaching a tag sequence to a RNA A gene sequence is hybridized with a forward primer which contains a T7 promoter, a tag sequence and Gene seq which is complementary with the gene sequence (A) A PCR results in a double stranded DNA that contains the gene sequence, the tag sequence and T7 promoter (B) An in vitro transcription reaction can be used to generate RNA that contains the coding region and the tag (C) The RNA can be used in vitro translation (D) The reverse primer for the PCR (A) contains both sequences for hybridizing with the gene sequence and a ribosome block sequence (Rblock) This block sequence can facilitate the retention of ribosome with the tagged RNA (D)

### DETAILED DESCRIPTION

Reference will now be made in detail to the preferred embodiments of the invention While the invention will be described in conjunction with the preferred embodiments, it will be understood that they are not intended to limit the invention to these embodiments On the contrary, the invention is intended to cover alternatives, modifications and equivalents, which may be included within the spirit and scope of the invention For example, high density oligonucleotide probe arrays are used as examples to describe many embodiments of the invention, however, the various aspects of the invention may not be limited to high density probe arrays All cited references, including patent and non-patent literature, are incorporated herein by reference in their entireties for all purposes

High density nucleic acid probe arrays, also referred to as DNA Microarrays, have become a method of choice for monitoring the expression of a large number of genes and for detecting sequence variations, mutations and polymorphism As used herein, Nucleic acids may include any polymer or oligomer of nucleosides or nucleotides (polynucleotides or oligonucleotidies), which include pyrimidine and purine bases, preferably cytosine, thymine, and uracil, and adenine and guanine, respectively See Albert L Lehninger, PRINCIPLES OF BIOCHEMISTRY, at 793-800 (Worth Pub 1982) and L Stryer BIOCHEMISTRY, 4 th Ed, (March 1995), both incorporated by reference Nucleic acids may include any deoxyribonucleotide, ribonucleotide or peptide nucleic acid component, and any chemical variants thereof, such as methylated, hydroxymethylated or glucosylated forms of these bases, and the like The polymers or oligomers may be heterogeneous or homogeneous in composition, and may be isolated from naturally-occurring sources or may be artificially or synthetically produced In addition, the nucleic acids may be DNA or RNA, or a mixture thereof, and may exist permanently or transitionally in single-stranded or double-stranded form, including homoduplex, heteroduplex, and hybrid states

As used herein, a probe is a molecule for detecting or binding a target molecule It can be any of the molecules in the same classes as the target referred to above A probe may refer to a nucleic acid, such as an oligonucleotide, capable of binding to a target nucleic acid of complementary sequence through one or more types of chemical bonds, usually through complementary base pairing, usually through hydrogen bond formation As used herein, a probe may include natural (I e A, G, U, C, or T) or modified bases (7-deazaguanosine, inosine, etc) In addition, the bases in probes may be joined by a linkage other than a phosphodiester bond, so long as the bond does not interfere with hybridization Thus, probes may be peptide nucleic acids in which the constituent bases are joined by peptide bonds rather than phosphodiester linkages Other examples of probes include antibodies used to detect peptides or other molecules, any ligands for detecting its binding partners When referring to targets or probes as nucleic acids, it should be understood that these are illustrative embodiments that are not to limit the invention in anyway

In preferred embodiments, probes may be immobilized on substrates to create an array An array may comprise a solid support with peptide or nucleic acid or other molecular probes attached to the support Arrays typically comprise a plurality of different nucleic acids or peptide probes that are coupled to a surface of a substrate in different, known locations These arrays, also described as “microarrays” or colloquially “chips” have been generally described in the art, for example, in Fodor et al, Science, 251 767-777 (1991), which is incorporated by reference for all purposes

Methods of forming high density arrays of oligonucleotides, peptides and other polymer sequences with a minimal number of synthetic steps are disclosed in, for example, U.S. Pat. Nos. 5,143,854, 5,252,743, 5,384,261, 5,405,783, 5,424,186, 5,429,807, 5,445,943, 5,510,270, 5,677,195, 5,571,639, 6,040,138, all incorporated herein by reference for all purposes The oligonucleotide analogue array can be synthesized on a solid substrate by a variety of methods, including, but not limited to, light-directed chemical coupling, and mechanically directed coupling See Pirrung et al, U.S. Pat. No. 5,143,854 (see also PCT Application No WO 90/15070) and Fodor et al, PCT Publication Nos WO 92/10092 and WO 93/09668, U.S. Pat. Nos. 5,677,195, 5,800,992 and 6,156,501 which disclose methods of forming vast arrays of peptides, oligonucleotides and other molecules using, for example, light-directed synthesis techniques See also, Fodor et al, Science, 251, 767-77 (1991) These procedures for synthesis of polymer arrays are now referred to as VLSIPS™ procedures Using the VLSIPS™ approach, one heterogeneous array of polymers is converted, through simultaneous coupling at a number of reaction sites, into a different heterogeneous array See, U.S. Pat. Nos. 5,384,261 and 5,677,195

Methods for making and using molecular probe arrays, particularly nucleic acid probe arrays are also disclosed in, for example, U.S. Pat. Nos. 5,143,854, 5,242,974, 5,252,743, 5,324,633, 5,384,261, 5,405,783, 5,409,810, 5,412,087, 5,424,186, 5,429,807, 5,445,934, 5,451,683, 5,482,867, 5,489,678, 5,491,074, 5,510,270, 5,527,681, 5,527,681, 5,541,061, 5,550,215, 5,554,501, 5,556,752, 5,556,961, 5,571,639, 5,583,211, 5,593,839, 5,599,695, 5,607,832, 5,624,711, 5,677,195, 5,744,101, 5,744,305, 5,753,788, 5,770,456, 5,770,722, 5,831,070, 5,856,101, 5,885,837, 5,889,165, 5,919,523, 5,922,591, 5,925,517, 5,658,734, 6,022,963, 6,150,147, 6,147,205, 6,153,743, 6,140,044 and D430024, all of which are incorporated by reference in their entireties for all purposes

Methods for signal detection and processing of intensity data are additionally disclosed in, for example, U.S. Pat. Nos. 5,547,839, 5,578,832, 5,631,734, 5,800,992, 5,856,092, 5,936,324, 5,981,956, 6,025,601, 6,090,555, 6,141,096, 6,141,096, and 5,902,723 Methods for array based assays, computer software for data analysis and applications are additionally disclosed in, e g, U.S. Pat. Nos. 5,527,670, 5,527,676,5,545,531, 5,622,829, 5,631,128, 5,639,423, 5,646,039, 5,650,268, 5,654,155, 5,674,742, 5,710,000, 5,733,729, 5,795,716, 5,814,450, 5,821,328, 5,824,477, 5,834,252, 5,834,758, 5,837,832, 5,843,655, 5,856,086, 5,856,104, 5,856,174, 5,858,659, 5,861,242, 5,869,244, 5,871,928, 5,874,219, 5,902,723, 5,925,525, 5,928,905, 5,935,793, 5,945,334, 5,959,098, 5,968,730, 5,968,740, 5,974,164, 5,981,174,5,981,185, 5,985,651, 6,013,440, 6,013,449, 6,020,135, 6,027,880, 6,027,894, 6,033,850, 6,033,860, 6,037,124, 6,040,138, 6,040,193, 6,043,080, 6,045,996, 6,050,719, 6,066,454, 6,083,697, 6,114,116, 6,114,122, 6,121,048, 6,124,102, 6,130,046, 6,132,580, 6,132,996 and 6,136,269, all of which are incorporated by reference in their entireties for all purposes

High-density polynucleotide probe arrays are among the most powerful and versatile tools for accessing the rapidly growing body of sequence information that is being generated by numerous public and private sequencing efforts Consequently, this technology is expected to have a major impact on the future of biological and biomedical research (Phimister B (Ed) (1999) Nat Genet Suppl 21 1, Schena R, Davis R W (2000) In Microarray Biochip Technology, Schena, M (ed), BioTechniques Books, Natick, M A, p 1)

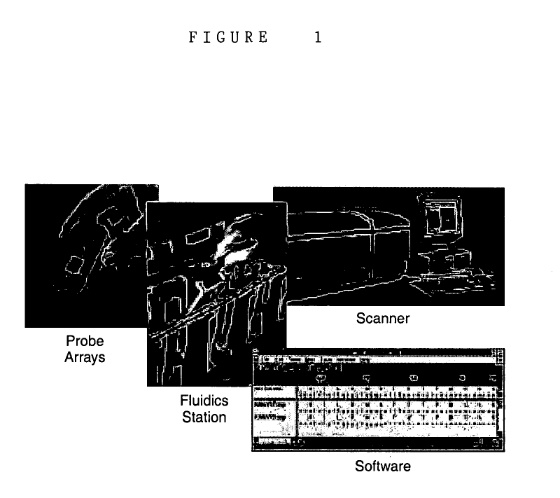

In a typical application, DNA or RNA target sequences of interest are isolated from a biological sample using standard molecular biology protocols The sequences are fragmented and labeled with fluorescent molecules for detection, and the mixture of labeled sequences is applied to the array, under controlled conditions, for hybridization with the surface probes The array is then imaged with a fluorescence-based reader to locate and quantify the binding of target sequences from the sample to complementary sequences on the array, and software reconstructs the sequence data and presents it in a format determined by the application Thus, in addition to the arrays themselves, the Affymetrix GeneChip® system provides a fluidics station for performing reproducible, automated hybridization and wash functions, a high-resolution scanner for reading the fluorescent hybridization image on the arrays, and software for processing and querying the data (FIG. 1)

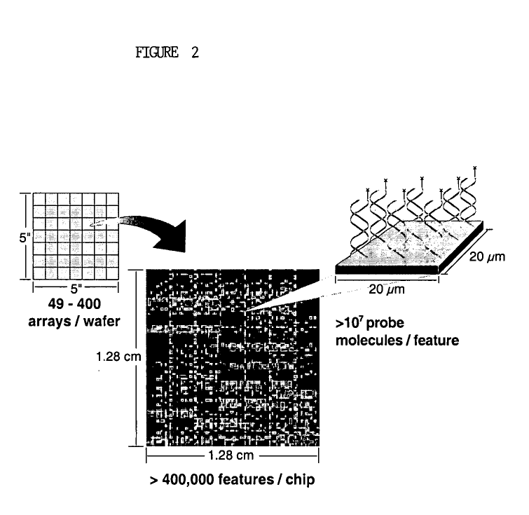

In some embodiments, oligonucleotide probe sequences are photolithographically synthesized, in a parallel fashion, directly on a glass substrate In a minimum number of synthesis steps, arrays containing hundreds of thousands of different probe sequences, 20-25 bases in length, can be generated at densities on the order of 10⁵-10⁶ sequences/cm² (FIG. 2)

Other technologies such as micropipetting or inkjet printing rely on mechanical devices to deliver minute quantitites of reagents to pre-defined regions of a substrate in a sequential fashion In contrast, the photolithographic synthesis process is highly parallel in nature, making it intrinsically robust and scalable This provides significant flexibility, and cost advantages in terms of materials management, manufacturing throughput, and quality control To researchers, the benefits are a high degree of reliability and uniformity of array performance, and an affordable price However, some aspects of the invention, particularly the applications of microarrays in various areas are not limited to any particular methods of manufacturing arrays

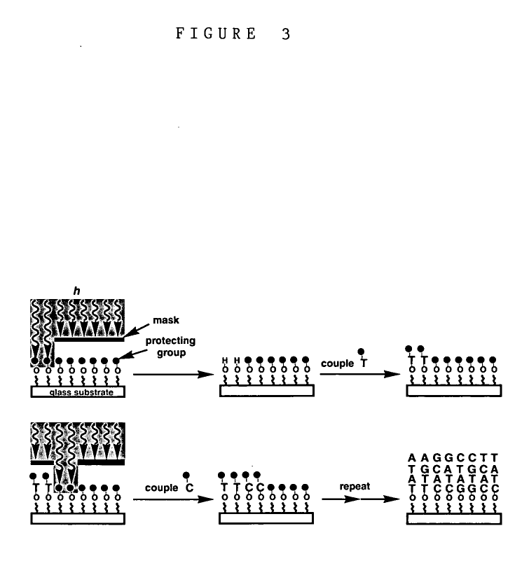

Light-directed synthesis (Fodor S P A, Read J L, Pirrung M C, Stryer L T, Lu A & Solas D (1991) Science 251 767, Pease A C, Solas D, Sullivan E J, Cronin M T, Holmes C P, Fodor S P A (1994) Proc Natl Acad Sci USA 91 5022, McGall G H, Barone A D, Diggelmann M, Fodor S P A, Gentalen E, Ngo N (1997) J Amer Chem Soc 119 5081) has made it possible to manufacture arrays containing hundreds of thousands of oligonucleotide probe sequences on glass chips little more than one cm² in size, and to do so on a commercial production scale In this process, 5′- or 3″-terminal protecting groups are selectively removed from growing oligonucleotide chains in pre-defined regions of a glass support, by controlled exposure to light through photolithographic masks (FIG. 3)

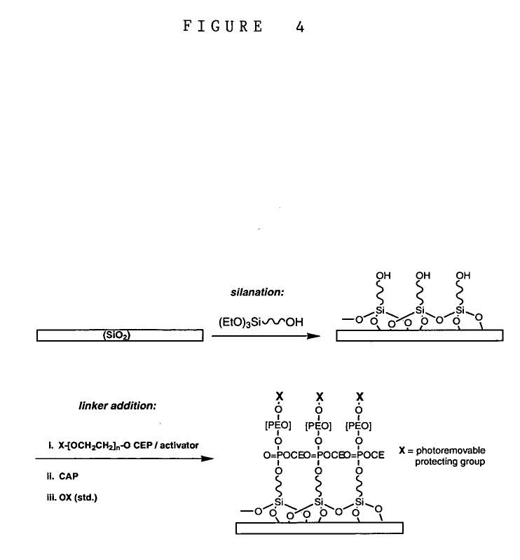

In some embodiments, prior to photolithographic synthesis, planar glass substrates are covalently modified with a silane reagent to provide a uniform layer of covalently bonded hydroxyalkyl groups on which oligonucleotide synthesis can be initiated (FIG. 4) In a second step, a photo-imagable layer is added by extending these synthesis sites with a poly(ethylene oxide) linker which has a terminal photolabile hydroxylprotecting group When specific regions of the surface are exposed to light, synthesis sites within these regions are selectively deprotected, and thereby activated for the addition of nucleoside phosphoramidite building blocks

These nucleotide precursors, also protected at the 5″ or 3″ position with a photolabile protecting group, are applied to the entire substrate, where they react with the surface hydroxyl groups in the pre-irradiated regions The monomer coupling step is carried out in the presence of a suitable activator, such as tetrazole or dicyanoimidazole The coupling reaction is followed by conventional capping and oxidation steps, which also use standard reagents and protocols for oligonucleotide synthesis (McGall G H, Barone A D, Diggelmann M, Fodor S P A, Gentalen E, Ngo N (1997) J Amer Chem Soc 119 5081, McGall G H, Fidanza J A (2001) In Rampal J B (ed) Methods in Molecular Biology DNA Arrays Methods and Protocols, Humana Press, Inc, Totowa, N.J., p 71) Alternating cycles of photolithographic deprotection and nucleotide addition are repeated to build the desired two-dimensional array of sequences as described in FIG. 3

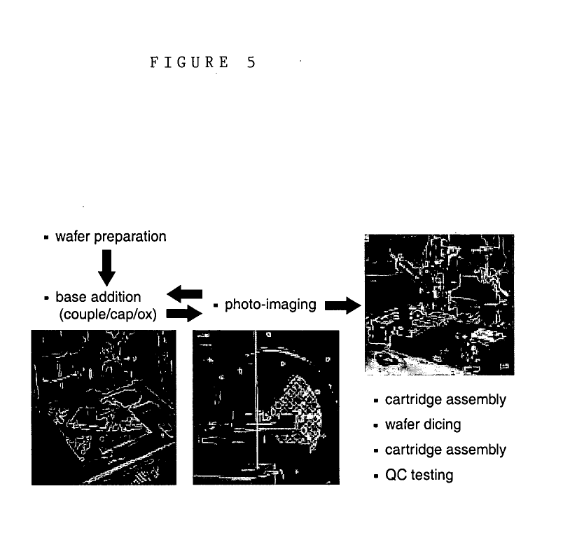

Semiautomated cleanroom manufacturing techniques, similar to those used in the microelectronics industry, have been adapted for the large-scale commercial production of GeneChip® arrays in a multi-chip wafer format (FIG. 5) Each wafer contains 49-400 replicate arrays, depending on the size of the array, and multiple-wafer lots can processed together in a procedure which takes less than 24 hours to complete Multiple lots are processed simultaneously on independent production synthesizers operating around the clock After a final chemical deprotection, finished wafers are diced into individual arrays, which are finally mounted in injection-molded plastic cartridges for single-use application (see FIG. 1)

The photolithographic process provides a very efficient route to high-density arrays by allowing parallel synthesis of large sets of probe sequences The number of required synthesis steps to fabricate an array is dependent only on the length of the probes, not the number of probes A complete set, or any subset, of probe sequences of length n requires at most, 4×n synthesis steps Masks can be designed to make arrays of oligonucleotide probe sequences for a variety of applications Most arrays are comprised of custom-designed sets of probes 20-25 bases in length, and optimized masking strategies allow such arrays to be completed in as few as 3 n steps

The spatial resolution of the photolithographic process determines the maximum achievable density of the array and therefore the amount of sequence information that can be encoded on a chip of a given physical dimension A contact lithography process (FIG. 3) is used to fabricate GeneChip® arrays with individual probe features that are 20×20 microns in size Between 49 and 400 identical arrays are produced simultaneously on 5×5 wafers For the largest-format chip currently in commercial production [1 6 cm²], this provides wafers of 49 individual arrays containing more than 400,000 different probe sequences each For arrays containing fewer probe sequences, this feature size enables more replicate arrays, up to 400, to be fabricated on each wafer The technology has proven capability for fabricating arrays with densities greater than 10⁶ sequences/cm², corresponding to features less than 10×10 microns in size This level of miniaturization is beyond the current reach of other technologies for array fabrication

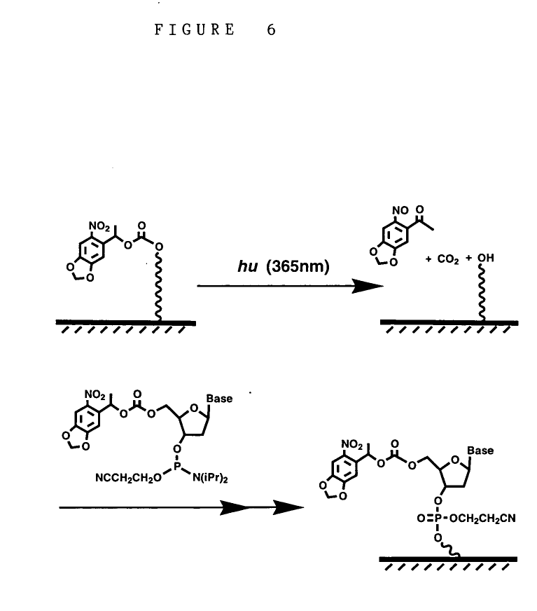

The current manufacturing process employs nucleoside monomers protected with a photo-removable 5′-(α-methyl-6-nitropiperonyloxycarbonyl), or MeNPOC group [4,5], depicted in (FIG. 6), which offers a number of advantages for large scale manufacturing These phosphoramidite monomers are relatively inexpensive to prepare, and photolytic deprotection is induced by irradiation at near-UV wavelengths (φ˜0 05, λₘₐₓ˜350 nm) so that photochemical modification of the oligonucleotides, which absorb energy at lower wavelengths, can be avoided The photolysis reaction involves an intramolecular redox reaction and does not require any special solvents, catalysts or coreactants Since the photolysis can be performed dry, high-contrast contact lithography can be used to achieve very high-resolution imaging Complete photo-deprotection requires less than one minute using filtered I-line (365+10 nm) emission from a commercial collimated mercury light source

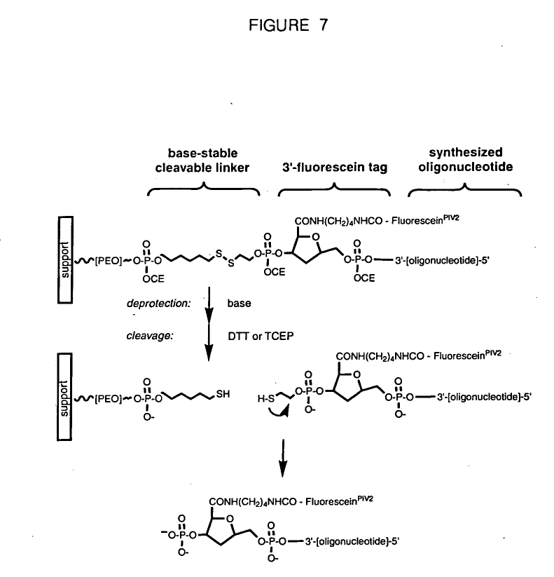

Photochemical deprotection rates and yields for oligonucleotide synthesis can both be monitored directly on planar supports using procedures based on surface fluorescence A sensitive assay has been developed in which test sequences are synthesized on a support designed to allow the cleavage and direct quantitative analysis of labeled oligonucleotide products using ion exchange HPLC with fluorescence detection (McGall G H, Barone A D, Diggelmann M (1999) Eur Pat Appl EP 967,217, Barone A D, Beecher J E, Bury P, Chen C, Doede T, Fidanza J A, McGall G H (2001) Nucleosides and Nucleotides) This method involves photolithographic synthesis of test sequences after the addition of a base-stable disulfide linker and a fluorescein monomer to the support (FIG. 7) The disulfide linker remains intact through synthesis and deprotection, but can be subsequently cleaved under reducing conditions to release the synthesis products, all of which are uniformly labelled with a 3″-fluorescein tag The labeled oligonucleotide synthesis products are then analysed using HPLC or capillary electrophoresis with fluorescence detection, enabling direct quantitative analysis of synthesis efficiency The sensitivity of fluorescence is a key feature of this methodology, since the quantities of DNA synthesis products on flat substrates are relatively low (1-100 pmole/cm²), and difficult to analyse accurately by other means

The average stepwise efficiency of light-directed oligonucleotide synthesis process is limited by the yield of the photochemical deprotection step which, in the case of MeNPOC nucleotides, is 90-94% The other chemical reactions involved in the base addition cycles (coupling, capping, oxidation) use reagents in a vast excess over surface synthesis sites, and provided that sufficient reagent concentrations and time are allowed for completion, they are essentially quantitative However, the sub-quantitative photolysis yields lead to incomplete or “truncated” probes, with the desired full-length sequences representing, in the case of 20-mer probes, approximately 10% of the total synthesis products

For a number of reasons, the presence of truncated probe impurities has a relatively minor impact on the performance characteristics of arrays when they are used for hybridization-based sequence analysis Firstly, the silanating agents used in this process provide an abundance of initial surface synthesis sites (>100 pmole/cm²), so that the absolute concentration of completed probes on the support remains high Thus, each of the 20×20 micron features on a typical array contains over 10⁷ full-length oligonucleotide molecules (FIG. 2) It should be noted that there is an optimum surface probe density for maximum hybridization signal and discrimination Thus, an increase in the synthesis yield through alternate chemistries or processes, while increasing the surface concentration of full-length probes, can actually reduce hybridization signal intensity This can be the result of steric and electrostatic repulsive effects which result when oligonucleotide molecules are spaced too closely together on the support Secondly, the truncated probes remain correct sequences, and any residual binding will be to the target sequences for which they were designed, albeit with slightly lower specificity Furthermore, array hybridizations are typically carried out under stringent conditions so that hybridization to significantly shorter (<n−4) oligomers is negligible Truncated sequences longer than n−4 are only about 10% as abundant as the full-length sequence, and contribute little to the total hybridization signal in a probe feature These factors, combined with the use of comparative intensity algorithms for data analysis, allow highly accurate sequence information to be “read” from these arrays with single-base resolution

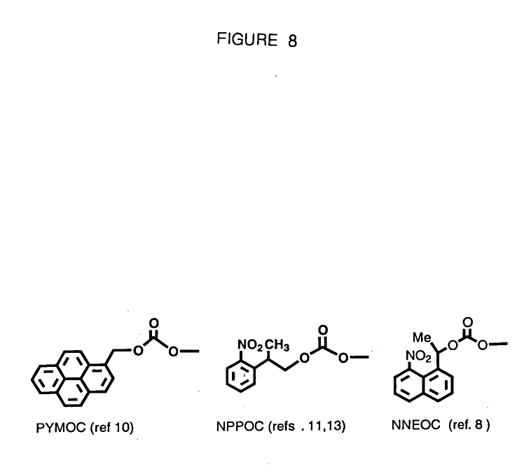

A number of alternate photolabile protecting groups have been described which may also be applicable to light-directed DNA array synthesis (McGall G H (1997) In Hon W (ed) Biochip Arrays IBC Library Series, Southboro, Mass., p 2 1, McGall G H, Nam N Q, Rava R (2000) U.S. Pat. No. 6,147,205, Hasan A, Stengele K-P, Giegrich H, Cornwell P, Isham K R, Sachleben R, Pfleiderer W, Foote R S (1997) Tetrahedron 53 4247, Pirrung M C, Fallon L, McGall G (1998) J Org Chem 63 241, Beier M, Hoheisel J D (2000) Nucleic Acids Res 28 e11) Some are capable of providing stepwise coupling yields in excess of 96%, and several examples are shown in FIG. 8

Some biochemical assay formats require probe array synthesis to proceed in the 5″-3″ direction so that the probes will be attached to the support at the 5″-terminus This can be achieved through the use of 3″-photo-activatable 5″-phosphoramidite building blocks (McGall G H, Fidanza J A (2001) In Rampal J B (ed) Methods in Molecular Biology DNA Arrays Methods and Protocols, Humana Press, Inc, Totowa, N.J., p 71)

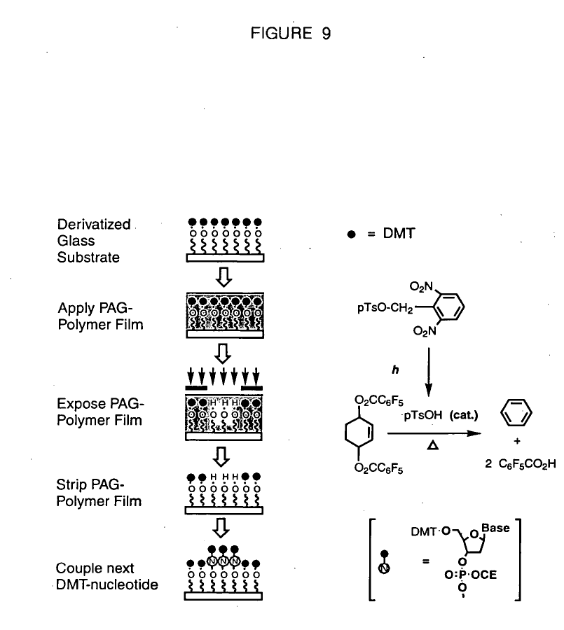

In some embodiments, photolithographic methods for fabricating DNA arrays which exploit polymeric photoresist films as the photoimageable component (McGall G, Labadie J, Brock P, Wallraff G, Nguyen T, Hinsberg W (1996) Proc Natl Acad Sci USA 93 13555, Wallraff G, Labadiej, Brock P, Nguyen T, Huynh T, Hinsberg W, McCall G (1997) Chemtech, February 22, Beecher J E, McCall G H, Goldberg M J (1997) Preprints Amer Chem Soc Div Polym Mater Sci Eng 76 597, Beecher J E, McGall G H, Goldberg M J (1997) Preprints Amer Chem Soc Div Polym Mater Sci Eng 77 394) are employed One of the advantages of the photoresist approach is that it can utilize conventional 4,4″-dimethoxytrityl (DMT)-protected nucleotide monomers These processes can also make use of chemical amplification of a photo-generated catalyst to achieve higher contrast and sensitivity (shorter exposure times) than conventional photo-removable protecting groups In this process, a polymeric thin film, containing a chemically amplified photo-acid generator (PAG), is applied to the glass substrate Exposure of the film to light creates localized development of an acid catalyst in the film adjacent to the substrate surface, resulting in direct removal of DMT protecting groups from the oligonucleotide chains (FIG. 9) This process has provided stepwise synthesis yields >98%, photolysis speeds an order of magnitude faster than that achievable with photoremovable protecting groups, and photolithographic resolution capability well below 10 microns This will enable the production of arrays with much higher information content than is currently attainable

In some additional embodiments, programmable digital micromirror devices, or digital light processors (DLPs) have been employed for photolithographic imaging, which offers a flexible approach to custom photolithographic array fabrication (U.S. Pat. No. 6,271,957, Singh-Gasson S, Green RD, Yue Y, Nelson C, Blattner F, Sussman M R, Cerrina F (1999) Nat Biotechnol 17 974) These devices were originally developed for digital image projection in consumer electronics products They are essentially high-density arrays of switchable mirrors which reflect light from a source into an optical system that focusses and projects the reflected image By using DLPs for photolithographic array synthesis, custom designs could be fabricated in a relatively short time, without the need for custom chrome-glass mask sets It should be noted that the standard lithographic approach using chrome-glass masks, which is ideal for mass producing standardized arrays, can also be adapted to the cost-effective production of smaller quantities of variable-content arrays This is achieved through the use of high-throughput mask design and fabrication capabilities, combined with new strategies which dramatically reduce the number of masks required to synthesize arrays

GeneChip® oligonucleotide probe arrays are used to access genetic information contained in both the RNA (gene expression monitoring) and DNA (genotyping) content of biological samples Many different GeneChip® products are now available for gene expression monitoring and genotyping complex samples from a variety of organisms The ability to simultaneously evaluate tens of thousands of different mRNA transcripts or DNA loci is transforming the nature of basic and applied research, and the range of application of DNA probe arrays is expanding at an accelerating pace

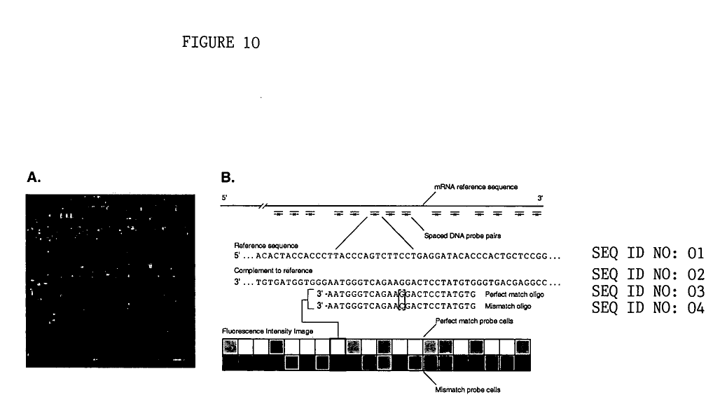

Currently, the most popular application for oligonucleotide microarrays is in monitoring cellular gene expression Standard GeneChip® arrays are encoded with public sequence information, but custom arrays are also designed from proprietary sequences FIG. 10 depicts how a gene expression array interrogates each transcript at multiple positions This feature provides more accurate and reliable quantitative information relative to arrays which use a single probe, such as a cDNA clone or PCR product, for each transcript Two probes are used at each targeted position of the transcript, one complementary (perfect match probe), and one with a single base mismatch at the central position (mismatch probe) The mismatch probe is used to estimate and correct for both background and signal due to non-specific hybridization The number of transcripts evaluated per probe array depends upon chip size, the individual probe feature size, and the number of probes dedicated to each transcript A standard 1 28×1 28 cm probe array, with individual 20×20 μm features, and 16 probe pairs per probe set, can interrogate approximately 12,000 transcripts This number is steadily increasing as manufacturing improvements shrink the feature size, and as improved sequence information and probe selection rules allow reductions in the number of probes needed for each transcript

Arrays are now available to examine entire transcriptomes from a variety of organisms including several bacteria, yeast, drosophila, arabidopsis, mouse, rat, and human Instead of monitoring the expression of a small subset of selected genes, researchers can now monitor the expression of all or nearly all of the genes for these organisms simultaneously, including a large number of genes of unknown function Numerous facets of biology and medicine are being explored using this powerful new capability Gene function has been explored in yeast by studying changes in expression levels throughout the cell cycle (Cho R J, Campbell M J, Winzeler E A, Steinmetz L, Conway A, Wodicka L, Wolfsberg T G, Gabrielian A E, Landsman D, Lockhart D J, Davis R W (1998) Mol Cell 2 65, Cho R J, Huang M, Dong H, Steinmetz L, Sapinoso L, Hampton G, Elledge S J, Davis R W, Lockhart D J, Campbell M J (2001) Nat Genet 27 48) Genetic pathways can be examined in great detail by monitoring the downstream transcriptional effects of inducing specific genes in cell culture, and the effects of drug treatment on gene expression levels can be surveyed (Debouck C, Goodfellow P N (1999) Nat Genet 21 4850) Expression arrays have also be used to screen thousands of genes to identify markers for human diseases such as cancer (Liotta L, Petricoin E (2000) Nature Reviews Genetics 1 48), muscular dystrophy (Chen Y W, Zhao P, Borup R, Hoffman E P (2000) J Cell Biol 151 1321), diabetes (Wilson S B, Kent S C, Horton H F, Hill A A, Bollyky P L, Hafler D A, Strominger J L, Byrne M C (2000) Proc Natl Acad Sci USA 97 7411), or for aging (Lee C K, Klopp R G, Weindruch R, Prolla T A (1999) Science 285 1390, Ly D H, Lockhart D J, Lerner R A, Schultz P G (2000) Science 287 2486)

One important area of research that is benefiting greatly from GeneChip® technology is cancer profiling, wherein gene expression monitoring is used to classify tumors in terms of their pathologies and responses to therapy Understanding the variation among cancers is the key to improving their treatment For example, a prostate tumor may be essentially harmless for twenty years in one patient, while an apparently similar tumor in another patient can be fatal within several months One patient's lymphoma may respond well to chemotherapy while another will not This variation of pathologies has motivated oncologists to assemble an impressive body of information to help classify tumors based on numerous histological, molecular, and clinical parameters This has required a massive effort by thousands of highly skilled and dedicated scientists over the past few decades

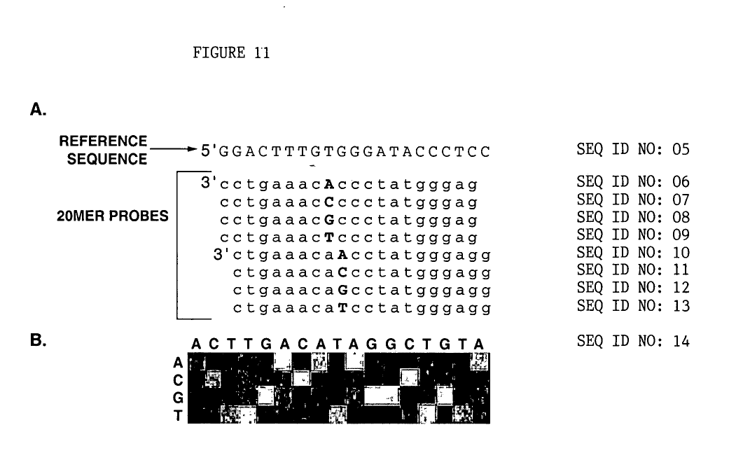

Oligonucleotide arrays are currently used primarily for two types of genotyping analysis Arrays for mutation or variant detection (FIG. 11) are used to screen sets of contiguous sequence for single-nucleotide differences Given a reference sequence, the basic design of genotyping arrays is quite simple four probes, varying only in the central position and each containing the reference sequence at all other positions, are made to interrogate each nucleotide of the reference sequence The target sequence hybridizes most strongly to its perfect complement on the array, which in most cases will be the probe corresponding to the reference sequence, but in the case of a nucleotide substitution, this will be one of the other three probes The other main type of genotyping performed with oligonucleotide arrays is SNP analysis, that is, the genotyping of biallelic single-nucleotide polymorphisms Because SNPs are the most common source of variation between individuals, they serve not only as landmarks to create dense genome maps but also as markers for linkage and loss of heterozygosity studies Large numbers of publicly available SNPs nearly one million to date have been found using gel-based sequencing as well as mutation detection arrays

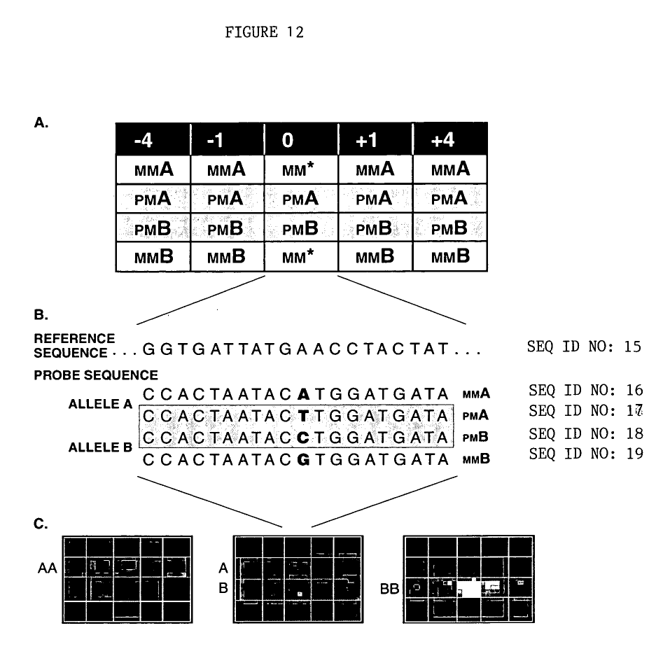

In addition to mutation detection arrays, at least two other types of oligonucleotide arrays can be used for SNP analysis The HuSNP assay allows nearly 1500 SNP-containing regions of the human genome to be amplified in just 24 multiplex PCRs and then hybridized to a single HuSNP array The SNPs cover all 22 autosomes and the X chromosome The probe strategy for a SNP array is shown in (FIG. 12) The probes for each SNP on the HuSNP array interrogate not only the two alleles of the SNP position, but also 3 or 4 positions flanking the SNP, the redundant data are of higher quality for the same reasons that the use of multiple probes improves gene expression monitoring array data

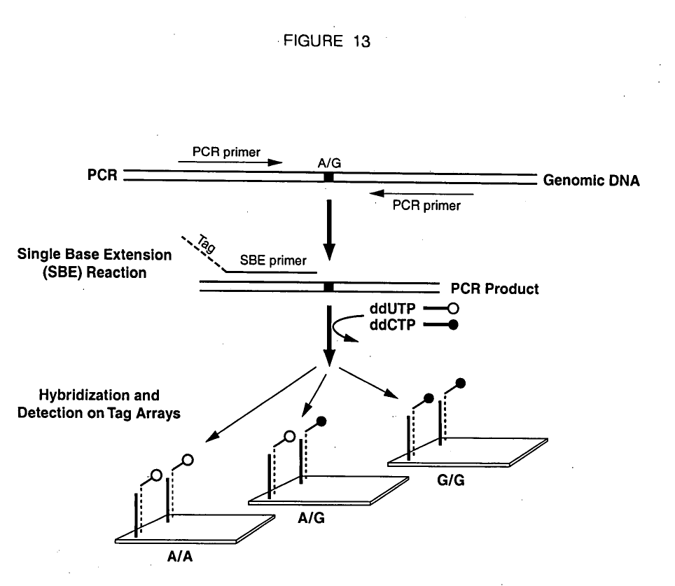

Although it is anticipated that the HuSNP assay will be appropriate for many applications, a more generic alternative is available in the form of the GenFlex™ array For this array, two thousand 20mer tag probe sequences were selected on the basis of uniform hybridization properties and sequence specificity The array includes 3 control probes for each tag (a complementary probe and single-base mismatch probes for both the tag and its complement) One way to use the GenFlex array for SNP analysis is illustrated in (FIG. 13) In this example, a single-base extension reaction is used, in which a primer abutting the SNP is extended by one base in the presence of the two possible dideoxy-NTPs, each of which is labeled with a different fluorophor Since each target-specific primer carries a different tag, the identity of each SNP is determined by hybridization of the single-base extension product to the corresponding tag probe in the GenFlex array The flexibility of the GenFlex approach lies in the freedom to partner any primer with any tag, a feature which enables other applications as well

While oligonucleotide arrays have been used primarily to determine the composition of RNA or DNA, many other applications are possible as well Any methodology that involves capturing large numbers of molecules that will hybridize to oligonucleotides can conceivably benefit from the highly parallel nature of these microarrays Furthermore, the hybridized molecules, which are essentially libraries, can serve as a platform for subsequent analyses based on biochemical reactions We describe below several recent non-traditional uses of GeneChip® arrays, and suggest a number of other potential applications as well

Tag arrays, such as the GenFlex array mentioned in the preceding section, have been used as molecular bar-code detectors In these experiments, mixtures of multiple yeast strains each carrying a unique tag in its genome and having a different gene deleted were subjected to a test such as drug treatment or growth in minimal medium, and then tag probe arrays were used to determine the proportion of each strain in the surviving population As in gene expression and genotyping applications, the molecular bar-coding strategy takes advantage of the ability of probe arrays to selectively bind many different sequences in a complex mixture simultaneously Parallel processing is not only faster and easier—it also minimizes the effect of variations in sample handling, thereby increasing the accuracy and precision of the measurements

There are many cases in which it is desirable to screen large numbers of proteins for a specific activity or function As genomic information rapidly identifies genes, there is an increasing desire to understand what these genes do, the burgeoning field of proteomics is devoted to just that issue Drug target investigation often involves testing for interactions between a drug and large panels of proteins Directed evolution projects create large libraries of mutated proteins that must be screened for desired new or altered activities

These undertakings typically require bacterial cloning and individual screening of thousands of clones In addition to the limitations on library size imposed by bacterial library construction, the need to handle and screen the clones creates a time and cost bottleneck and can reduce the ultimate success of the project

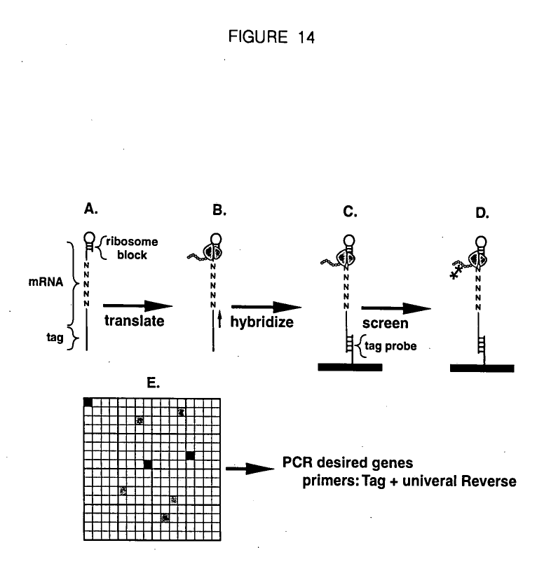

In one aspect of the invention, methods are provided for the use of microarrays for proteomics and other protein screening applications For example, by attaching a different oligonucleotide sequence tag to each member of a group of proteins to be analyzed, hybridization would allow them to bearrayed in discreet locations on a chip for parallel screening Proteins of interest would be identified by their position on the array In one examplary approach (FIG. 14), the tag is attached to the protein genetically by linking the tag to the mRNA and then translating the protein in such a manner that the protein remains associated with the mRNA, as is done in ribosome display to create and capture high affinity antibodies (Hanes J, Jermutus L, Pluckthun A (2000) Methods Enzymol 328 404)

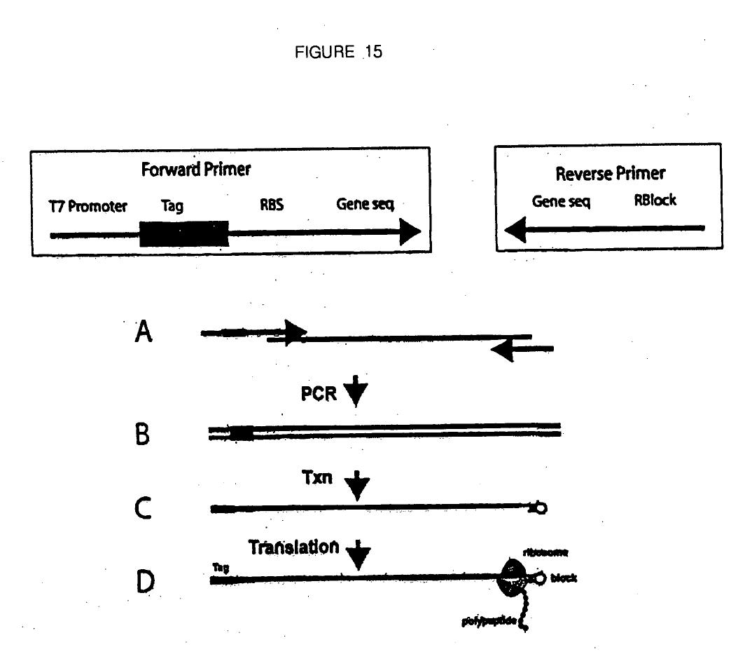

A unique tag sequence can be attached to each target (mRNA, cDNA, gene, DNA fragment) in several ways One method, depicted in FIG. 15, incorporates a tag in a target-specific PCR primer, in this example, the forward primer The forward primer for each target is assigned a different tag Tagging n targets thus requires n different forward primers, the reverse primers can be either target-specific as in the example, or common to all targets if the targets have common ends, for example polyA tracts or adaptor attachments Each target can be tagged in a separate PCR, or multiple reactions can be done in the same vessel, i e, multiplex As the figure depicts, additional features for transcribing and translating the target can be incorporated into the PCR primers

In another examplary embodiment, a unique tag is assigned to each target without using target-specific primers This operationally simpler tagging can be accomplished by using significantly more tags than targets For example, a pool of 10,000 targets can be combined with a pool of 1,000,000 tags to ensure that nearly every target receives a different tag The tags can be part of a primer pool The primers in the pool consist of at least two functional parts the 3′ portion of each primer in the pool is the same, and anneals to an end common to all the targets, 5′ to this common region of the primer is a tag sequence that varies among the members of the primer pool, 5′ to the tag sequence can be additional sequence, for example, to encode transcriptional or translational signals After annealing the primer pool to the target pool, the primers are extended to make a copy of each target Amplification of the extended primer can then be done During amplification care must be used not to attach new tags to targets, for example, by using the same primer pool that was used for the initial annealing/extension event that assigned tags to targets Retagging can be avoided by using an amplification primer that anneals 5′ to the tags

The tags can also be carried on adaptor nucleic acid molecules that are ligated to the target pool Again, nearly unique tagging can be accomplished by using a significantly larger number of different tags than targets Likewise, the tag library can be built into a plasmid pool that contains significantly more members than does the target pool (see, for example, Brenner, et al (2000) Proc Natl Acad Sciences 97 1665)

In some cases it is not necessary for each different target to have a unique tag For example, in screening a library of protein variants, as depicted in FIG. 14, in some cases it is acceptable for multiple variants to travel to the same address on the array During screening the output signal from such an address is less pure than from an address with just one variant, and potential high signal can be diluted, but this drawback can be an acceptable trade-off depending on other conditions and throughput requirements Subsequent amplification of the targets on such an address can capture undesired variants, but an additional subsequent retagging and rescreening of all the captured variants makes it unlikely that the same unwanted variant is captured again In other words, in some cases it can be more efficient to retag and rescreen than to require unique tags for each target

Ribsome display is a method has been developed in which whole functional proteins can be enriched in a cell-free system for their binding function, without the use of any cells, vectors, phages or transformation (Proc Natl Acad Sci 94, 4937, 1997, Curr Opin Biotechnol 9, 534, 1998, Curr Top Microbiol Immunol, 243, 107, 1999, J Immunol Meth 231, 119, 1999, FEBS Lett, 450, 105, 1999) This technology is based on in vitro translation, in which both the mRNA and the protein product do not leave the ribosome This results in two fundamental advantages (i) the diversity of a protein library is no longer restricted by the transformation efficiency of the bacteria, and (ii), because of the large number of PCR cycles, errors can be introduced, and by the repeated selection for ligand binding, improved molecules are selected Correctly folded proteins can be selected, if the folding of the protein on the ribosome is secured (Nat Biotechnol 15, 79, 1997)

The protein-mRNA-tag complex is hybridized to the tag probe array, and screened for protein activity on the array The proteins could be translated on the array, after hybridization Genes of interest are recovered, either directly from the array or from another aliquot of the mRNA library, by PCR using the tag sequence for one primer and a common 3″ end sequence as the other primer

One use for such a system would be in directed evolution projects in which large gene libraries are made by cloning into cells, usually bacteria or yeast, followed by propagating and screening each clone individually for production of a protein with new or improved properties The tag system would not only eliminate the need to transform and handle individual clones but would also allow highly parallel screening because thousands of variants could be assayed simultaneously on the same array Another use for the tag system would be to screen (poly) peptides made from existing mRNA molecules for properties such as drug binding For example, all the mRNAs from a pathogenic bacterial strain could be converted to tagged proteins, which could then be screened for the ability to bind antibiotic candidates The RNA molecules themselves could also be screened, as some drugs act directly on RNA In a preferred embodiment, the oligonucleotide tag is added directly to proteins, a method that might be useful in cases in which clones are already separated and one wishes to use the tag probe array only for parallel screening

It is to be understood that the above descripis intended to be illustrative and not restrictive Many variations of the invention will be apparent to those of skill in the art upon reviewing the above description All cited references, including patent and non-patent literature, are incorporated herein by reference in their entireties for all purposes

1. A method for screening a plurality of polypeptides comprising
linking each of the plurality of polypeptides with a nucleic acid tag to obtain tagged polypeptides,
hybridizing the tagged polypeptides with an oligonucleotide probe array to immobilize the tagged polypeptides on the array, wherein the oligonucleotide probe array has at least one probe for each of the nucleic acid tag, and screening the polypeptides for an activity

2. The method of claim 1 wherein the linking comprises
attaching oligonucleotide tags to a plurality of mRNAs, and
translating the mRNAs to produce the plurality of polypeptides, wherein the translation is performed under the condition that the resulting peptides are attached to the mRNA

3. The method of claim 2 wherein each of the mRNAs is attached with a different tag

4. The method of claim 3 wherein the screening comprises determining the binding affinity of the immobilized polypeptides with a ligand

5. The method of claim 4 wherein the ligand is a drug candidate

6. The method of claims 2, 3, 4, 5 or 6 wherein the oligonucleotide probe array has at least 400 different oligonucleotide probes per cm²

7. The method of claims 2, 3, 4, 5 or 6 wherein the oligonucleotide probe array has at least 1000 different oligonucleotide probes per cm²

8. The method of claims 2, 3, 4, 5 or 6 wherein the oligonucleotide probe array has at least 10000 different oligonucleotide probes per cm²

9. The method of claims 2, 3, 4, 5 or 6 wherein the plurality of polypeptides comprise at least 50 polypeptides

10. The method of claims 2, 3, 4, 5 or 6 wherein the plurality of polypeptides comprise at least 100 polypeptides

11. The method of claims 2, 3, 4, 5 or 6 wherein the plurality of polypeptides comprise at least 1000 polypeptides

12. A method for screening a plurality of polypeptides comprising
attaching oligonucleotide tags to a plurality of mRNAs,
hybridizing the plurality of mRNAs to an oligonucleotide array, wherein the oligonucleotide array has at least one probe for each of the oligonucleotide tags,
translating the mRNAs to produce the plurality of polypeptides, wherein the translation is performed under the condition that the resulting peptides are attached to the mRNA, and
screening the polypeptides for an activity

13. The method of claim 12 wherein each of the mRNAs is attached with a different tag

14. The method of claim 13 wherein the screening comprises determining the binding affinity of the immobilized polypeptides with a ligand

15. The method of claim 14 wherein the ligand is a drug candidate

16. The method of claims 13, 14, or 15 wherein the oligonucleotide probe array has at least 400 different oligonucleotide probes per cm²

17. The method of claims 13, 14, or 15 wherein the oligonucleotide probe array has at least 1000 different oligonucleotide probes per cm²

18. The method of claims 13, 14, or 15 wherein the oligonucleotide probe array has at least 10000 different oligonucleotide probes per cm²

19. The method of claims 13, 14, or 15 wherein the plurality of polypeptides comprise at least 50 polypeptides

20. The method of claims 13, 14, or 15 wherein the plurality of polypeptides comprise at least 100 polypeptides

21. The method of claims 13, 14, or 15 wherein the plurality of polypeptides comprise at least 1000 polypeptides


SEQUENCE LISTING (19 sequences)

SEQ ID NO: 1 - DNA, 49 bp, Artificial
  Synthetic DNA
acactaccacccttacccagtcttcctgaggatacacccactgctccgg

SEQ ID NO: 2 - DNA, 49 bp, Artificial
  Synthetic DNA
tgtgatggtgggaatgggtcagaaggactcctatgtgggtgacgaggcc

SEQ ID NO: 3 - DNA, 25 bp, Artificial
  Synthetic DNA
aatgggtcagaaggactcctatgtg

SEQ ID NO: 4 - DNA, 25 bp, Artificial
  Synthetic DNA
aatgggtcagaacgactcctatgtg

SEQ ID NO: 5 - DNA, 21 bp, Artificial
  Synthetic DNA
ggactttgtgggataccctcc

SEQ ID NO: 6 - DNA, 20 bp, Artificial
  Synthetic DNA
cctgaaacaccctatgggag

SEQ ID NO: 7 - DNA, 20 bp, Artificial
  Synthetic DNA
cctgaaaccccctatgggag

SEQ ID NO: 8 - DNA, 20 bp, Artificial
  Synthetic DNA
cctgaaacgccctatgggag

SEQ ID NO: 9 - DNA, 20 bp, Artificial
  Synthetic DNA
cctgaaactccctatgggag

SEQ ID NO: 10 - DNA, 20 bp, Artificial
  Synthetic DNA
ctgaaacaacctatgggagg

SEQ ID NO: 11 - DNA, 20 bp, Artificial
  Synthetic DNA
ctgaaacaccctatgggagg

SEQ ID NO: 12 - DNA, 20 bp, Artificial
  Synthetic DNA
ctgaaacagcctatgggagg

SEQ ID NO: 13 - DNA, 20 bp, Artificial
  Synthetic DNA
ctgaaacatcctatgggagg

SEQ ID NO: 14 - DNA, 17 bp, Artificial
  Synthetic DNA
acttgacataggctgta

SEQ ID NO: 15 - DNA, 20 bp, Artificial
  Synthetic DNA
ggtgattatgaacctactat

SEQ ID NO: 16 - DNA, 20 bp, Artificial
  Synthetic DNA
ccactaatacatggatgata

SEQ ID NO: 17 - DNA, 20 bp, Artificial
  Synthetic DNA
ccactaatacttggatgata

SEQ ID NO: 18 - DNA, 20 bp, Artificial
  Synthetic DNA
ccactaatacctggatgata

SEQ ID NO: 19 - DNA, 20 bp, Artificial
  Synthetic DNA
ccactaatacgtggatgata